(19) United States
(12) Patent Application Publication
(10) Pub. No.: US 2020/0276436 A1
(43) Pub. Date: Sep. 3, 2020

(54) METHOD FOR TREATING SEPSIS AND SEPTIC SHOCK

(71) Applicant: VasoActiv Biomedical Technologies LLC, Tulsa, OK (US)

(72) Inventor: John M. Owen, Tulsa, OK (US)

(21) Appl. No.: 16/805,364

(22) Filed: Feb. 28, 2020

Related U.S. Application Data
(60) Provisional application No. 62/811,868, filed on Feb. 28, 2019.

(51) Int. Cl. A61N 1/04

(52) U.S. Cl. CPC A61N 1/0492; A61N 1/0456; A61N 1/0452

(57) ABSTRACT

A method and system for treating sepsis/septic shock by increasing whole body blood flow. A method and protocol for application of neuromuscular electrical stimulation (NMES) to the skeletal muscles using a plurality of treatment pads (electrodes) on one or more limbs, activated sequentially, with overlapping timing, distal to proximal, with the impulses released in a similar, following pattern to enhance the refill cycle, resulting in accelerated wave-form blood flow in cardiovascular circulation.

### CROSS-REFERENCE TO RELATED CASES

This application claims the benefit of U.S. provisional patent application Ser. No. 62/811,868, filed on Feb. 28, 2019, and incorporates such provisional application by reference into this disclosure as if fully set out at this point.

### FIELD OF THE INVENTION

This disclosure relates to medical treatment for a patient with sepsis or septic shock.

### BACKGROUND OF THE INVENTION

Sepsis is a serious medical condition and the leading cause of death in U.S. hospitals. More than 1.5 million people get sepsis each year in the U.S., and 270,000 people die from sepsis every year—one every two minutes—more than prostate cancer, breast cancer and AIDS combined. One third of patients who die in hospitals have sepsis, and sepsis hospitalization in the U.S. consumes more than $27 billion each year.

Sepsis is caused by an overwhelming immune response to infection. The body releases immune chemicals into the blood to combat the infection. Those chemicals trigger widespread inflammation, which leads to blood clots and leaky blood vessels. As a result, blood circulation is impaired or collapses, and that deprives organs of nutrients and oxygen which can lead to organ damage and death.

Early diagnosis is critical. Each hour of delay in the beginning of treatment increases the risk of death by 8%. Doctors typically treat people with sepsis in hospital intensive care units where they try to stop the infection, protect the vital organs, and prevent a drop in blood pressure. This almost always includes the use of antibiotic medications, vasopressors and IV fluids. As the disease progresses, the patient can experience a drop in blood pressure because their body has introduced large quantities of vasodilators into their bloodstream. Blood pools in the veins, circulation collapses and organs can begin to fail for lack of oxygen and nutrients. Commonly, vasopressors are administered to constrict blood vessels in the extremities so more blood will collect in the body's core to protect the patient's vital organs. This sometimes results in the amputation of the extremities and elevates mortality.

Disseminated intravascular coagulation, or DIC, is a complicated condition that can occur when someone has severe sepsis or septic shock. Both blood clotting and difficulty with clotting may occur, causing a vicious cycle. Small blood clots can develop throughout your bloodstream, especially in the microscopic blood vessels called capillaries, blocking the blood flow to many parts of your body, including your limbs and your organs. This blood flow is needed to bring oxygen and nutrients to the tissues. On the reverse side of the cycle, DIC can cause increased bleeding because the body is using up so many of the blood clotting proteins for the multiple blood clots in the blood vessels that there are not enough of them left to clot the blood elsewhere.

There are several medical conditions that can cause DIC, including sepsis. DIC affects about 35% of patients who have sepsis. Sometimes incorrectly called blood poisoning, sepsis is the body's often deadly response to infection. Sepsis and septic shock can result from an infection anywhere in the body, such as pneumonia, influenza, or urinary tract infections. Many who do survive sepsis are left with life-changing effects, such as post-traumatic stress disorder, chronic pain and fatigue, organ dysfunction and/or amputations.

More seriously affected patients might need a breathing tube, kidney dialysis, or surgery to remove an infection. Despite years of research, scientists have not yet developed a solution that specifically targets the aggressive immune response seen with sepsis. The fact that 15% to 50% of sepsis patients die indicates that there is a need for more effective treatments.

### SUMMARY OF THE INVENTION

Embodiments of the present disclosure provides methods of Septic Shock Therapy (SST). In various embodiments the methods include elevation of blood movement, raising of shear stress, and augmented endothelial mechanotransduction. Some embodiments use a device that generates electrical waveforms capable of contracting muscle. The device may be attached by lead wires to pairs of treatment pads (also called electrodes) in 180 degree opposition to each other. These pairs of treatment pads may be placed on a patient at 16 locations (4 per extremity). Short bursts of neuromuscular stimulation may be applied in an overlapping, sequential manner (e.g., distal to proximal). This process will effectively “milk” blood from the extremities and return it to the heart, resulting in higher venous return and cardiac output. The increased cardiac output supplies more blood to the body's core organ area. In addition to the basic benefits of blood movement, which is fibrinolytic, and the delivery of oxygen, which is needed for tissue viability and white blood cell removal of bacteria, the flowing blood elevates endothelial mechanotransduction and the autocrine and paracrine systems. This leads to the increased production of several helpful substances such as prostacyclin, thrombomodulin, glutathione, activated protein C, protein S and others.

Embodiments of the present disclosure via the combination of effects described, and possibly others, stabilizes the patient providing more time so that other medicines and procedures in the protocol may bring about a cure or remission.

The invention of the present disclosure, in one aspect thereof, comprises a method of treatment of sepsis in a patient in need of such treatment. The method includes applying a first plurality of pairs of electric treatment pads to a first limb of the patient from a distal to a proximal location on the first limb, and providing an electrical neuromuscular stimulation to the first plurality of pairs of treatment pads according to a waveform. The waveform is applied in a sequential and overlapping manner to the first plurality of pairs of treatment pads such that the electrical neuromuscular stimulation progresses from the distal to the proximal location on the first limb. The waveform activates a first most distal pair of pads of the first plurality of treatment pads and thereafter activates a second most distal pair of pads of the first plurality of treatment pads while keeping the first most distal pair of pads of the first plurality of treatment pads activated. Finally, the waveform deactivates the first most distal pair of pads of the first plurality of treatment pads when a third most distal pair of pads of the first plurality of treatment pads is activated.

The method may also include applying a second plurality of pairs of electric treatment pads to a second limb of the patient from a distal to a proximal location on the second limb, and providing the electrical neuromuscular stimulation to the second plurality of pairs of treatment pads according to the predetermined waveform. The waveform is applied in a sequential and overlapping manner to the second plurality of pairs of treatment pads such that the electrical neuromuscular stimulation progresses from the distal to the proximal location on the second limb. The waveform activates a first most distal pair of pads of the second plurality of treatment pads and thereafter activates a second most distal pair of pads of the second plurality of treatment pads while keeping the first most distal pair of pads of the second plurality of treatment pads activated. As before, the waveform deactivates the first most distal pair of pads of the second plurality of treatment pads when a third most distal pair of pads of the second plurality of treatment pads is activated.

The method of may further include applying a third plurality of pairs of electric treatment pads to a third limb of the patient from a distal to a proximal location on the third limb, and providing the electrical neuromuscular stimulation to the third plurality of pairs of treatment pads according to the predetermined waveform. The waveform is applied in a sequential and overlapping manner to the third plurality of pairs of treatment pads such that the electrical neuromuscular stimulation progresses from the distal to the proximal location on the third limb. The waveform activates a first, most distal pair of pads of the third plurality of treatment pads and thereafter activates a second most distal pair of pads of the third plurality of treatment pads while keeping the first most distal pair of pads of the third plurality of treatment pads activated. The waveform deactivates the first most distal pair of pads of the third plurality of treatment pads when a third most distal pair of pads of the third plurality of treatment pads is activated.

The method may further include applying a fourth plurality of pairs of electric treatment pads to a fourth limb of the patient from a distal to a proximal location on the fourth limb, and providing the electrical neuromuscular stimulation to the fourth plurality of pairs of treatment pads according to the predetermined waveform. The waveform is applied in a sequential and overlapping manner to the fourth plurality of pairs of treatment pads such that the electrical neuromuscular stimulation progresses from the distal to the proximal location on the fourth limb. The waveform activates a first most distal pair of pads of the fourth plurality of treatment pads and thereafter activates a second most distal pair of pads of the fourth plurality of treatment pads while keeping the first most distal pair of pads of the fourth plurality of treatment pads activated. Finally, as with the other limbs, the waveform deactivates the first most distal pair of pads of the fourth plurality of treatment pads when a third most distal pair of pads of the fourth plurality of treatment pads is activated.

In some embodiments, the first limb is an arm. The first limb may also be a leg. In other cases the first limb and second limb are arms but the first limb and second limb can be legs. The first limb may be a leg and the second limb an arm. In some cases the first and second limbs are left and right arms, respectively, and the third and fourth limbs are left and right legs, respectively. In such case, the waveform may applied to the first and third plurality of treatment pads simultaneously, followed by application of the waveform to the second and fourth treatment pads simultaneously. In some cases application of the waveform to the first and third plurality of treatment pads does not overlap with application of the waveform to the second and fourth plurality of treatment pads. The electrical neuromuscular stimulation at each pair of the first plurality of pairs of treatment pads may be about 500 ms in duration.

The invention of the present disclosure, in another aspect thereof, comprises a method of treatment of a patient in need of a treatment for sepsis. The method includes applying a first plurality of electrically conductive treatment pads along plurality of locations along a first limb of the patient in need of treatment, the locations along the first limb being from distal to proximal, and providing an electrical waveform as an application of current to the first plurality of electrically conductive treatment pads. The electrical waveform is applied in a sequential and overlapping manner to the first plurality of treatment pads such that an electrical neuromuscular stimulation progresses from the distal to proximal locations on the first limb. The electrical waveform applies the electrical neuromuscular stimulation such that stimulation occurs at two adjacent locations on the first limb, except for a beginning of the waveform when only a most distal location of the first limb receives stimulation and an end of the waveform when only a most proximal location on the first limb receives stimulation. The electrical waveform maintains stimulation at no more than two adjacent locations on the first limb at any time.

The previous method may also include applying a second plurality of electrically conductive treatment pads along plurality of locations along a second limb of the patient in need of treatment, the locations along the first limb being from distal to proximal, and providing the electrical waveform as an application of current to the second plurality of electrically conductive treatment pads. The electrical waveform is applied in the sequential and overlapping manner to the second plurality of treatment pads such that an electrical neuromuscular stimulation progresses from the distal to proximal locations on the second limb. The electrical waveform applies the electrical neuromuscular stimulation such that stimulation occurs at two adjacent locations on the second limb, except for a beginning of the waveform when only a most distal location of the second limb receives stimulation and an end of the waveform when only a most proximal location on the second limb receives stimulation. Again, the electrical waveform maintains stimulation at no more than two adjacent locations on the second limb at any time.

In such cases, the first limb may be an arm of the patient and the second limb a leg of the patient. The electrical waveform may be applied to the plurality of electrically conductive treatment pads on the first limb and to the plurality of electrically conductive treatment pads on the second limb simultaneously.

The invention of the present disclosure, in another aspect thereof, comprises a device for treatment of a patient in need of a treatment for sepsis. The device includes an electrical waveform generator, a first plurality of pairs of electric treatment pads adapted to attach to a first limb of the patient from a distal to a proximal location on the first limb, each of the pairs of electrical treatment pads of the first plurality of pairs of electrical treatment pads, when active, providing electrical neuromuscular stimulation at their respective location to the first limb of the patient, and leads electrically connecting the first plurality of pairs of electric treatment pads to the waveform generator. The electrical waveform generator provides a stimulation waveform to the first plurality of pairs of electrical treatment pads and the stimulation waveform activates the first plurality of pairs of treatment pads in a sequential and overlapping manner such that the electrical neuromuscular stimulation progresses from the distal to the proximal location on the first limb. The stimulation waveform also activates a first most distal pair of pads of the first plurality of treatment pads and thereafter activates a second most distal pair of pads of the first plurality of treatment pads while keeping the first most distal pair of pads of the first plurality of treatment pads activated. The stimulation waveform deactivates the first most distal pair of pads of the first plurality of treatment pads when a third most distal pair of pads of the first plurality of treatment pads is activated.

The device may also include a second plurality of pairs of electric treatment pads adapted to attach to a second limb of the patient from a distal to a proximal location on the second limb, each of the pairs of electrical treatment pads of the second plurality of pairs of electrical treatment pads, when active, providing electrical neuromuscular stimulation at their respective locations to the second limb of the patient, and leads electrically connecting the second plurality of pairs of electric treatment pads to the waveform generator. The electrical waveform generator provides the stimulation waveform to the second plurality of pairs of electrical treatment pads. The stimulation waveform activates the second plurality of pairs of treatment pads in a sequential and overlapping manner such that the electrical neuromuscular stimulation progresses from the distal to the proximal location on the second limb. The stimulation waveform activates a first most distal pair of pads of the second plurality of treatment pads and thereafter activates a second most distal pair of pads of the second plurality of treatment pads while keeping the first most distal pair of pads of the first plurality of treatment pads activated. The stimulation waveform deactivates the first most distal pair of pads of the second plurality of treatment pads when a third most distal pair of pads of the second plurality of treatment pads is activated.

In some embodiments, the waveform generator provides the stimulation waveform to the first and second pluralities of treatment pads simultaneously.

### BRIEF DESCRIPTION OF THE DRAWINGS

FIG. 1 is a schematic drawing showing a blood vessel and plurality treatment pads prior to activation according to aspects of the present disclosure.

FIG. 2 is a schematic drawing showing the blood vessel and pads of FIG. 1 at initiation of a treatment sequence.

FIG. 3 is a schematic drawing showing the blood vessel and pads of FIG. 1 as treatment continues from FIG. 2.

FIG. 4 is a schematic drawing showing the blood vessel and pads of FIG. 1 as treatment continues from FIG. 3.

FIG. 5 is a schematic drawing showing the blood vessel and pads of FIG. 1 as treatment continues from FIG. 4.

FIG. 6 is a schematic drawing showing the blood vessel and pads of FIG. 1 as treatment continues from FIG. 5.

FIG. 7 is a schematic drawing showing the blood vessel and pads of FIG. 1 as treatment continues from FIG. 6.

FIG. 8 is a drawing of a wave-form stimulation device for providing treatments according to aspects of the present disclosure.

FIG. 9 is a stylized representation of the human vascular system (left) and a stylized representation of the blood flow resulting from the systems and methods of the present disclosure (right).

FIG. 10 is a graphic representation of some endothelial mechanotransduction-generated changes in body chemistry via autocrine and paracrine processes.

FIG. 11 is a representation of a human body illustration potential approximate placement of treatment pads according to aspects of the present disclosure.

FIG. 12 is a flow chart showing various aspects and benefits according to aspects of the present disclosure.

### DETAILED DESCRIPTION OF THE PREFERRED EMBODIMENTS

One of the main reason sepsis and septic shock are such serious conditions, is that there is a narrow window in which to apply a remedy before the patient is beyond help. Once the blood pressure drops and circulation slows to a crawl, organ failure can happen within hours. For people in late stage severe sepsis, the mortality rate is 50%.

Circulatory collapse, which leads to multi-organ failure, is the gravest complication of sepsis. Another serious problem called DIC (disseminated intravascular coagulopathy) causes both bleeding and microscopic clotting in all the tiny blood vessels of the body, which can lead to gangrene in limbs indicating tissue death from low flow and poor oxygenation of all tissues.

The systems and methods of the present disclosure may artificially increase blood flow using a series of neuromuscular stimulations which are timed in overlapping, sequential order from distal to proximal. The stimulation uses a waveform capable of eliciting tetanic contractions. In various embodiments, the stimulation relies on contraction of a patient's own skeletal muscle surrounding the veins to promote return circulation from the extremities which, for various reasons, counteracts sepsis.

Mechanistically, inflammation modulates thrombotic responses by upregulating procoagulants, downregulating anticoagulants and suppressing fibrinolysis. Blood flow and shear stress, on the other hand as effected by methods and systems of the present disclosure, does the exact opposite. Treatments according to the present disclosure downregulate procoagulants, upregulates anticoagulants, and boosts fibrinolysis. Thus the current disclosure provides various embodiments of systems and methods that mitigate inflammation and coagulation through these flow-dependent processes.

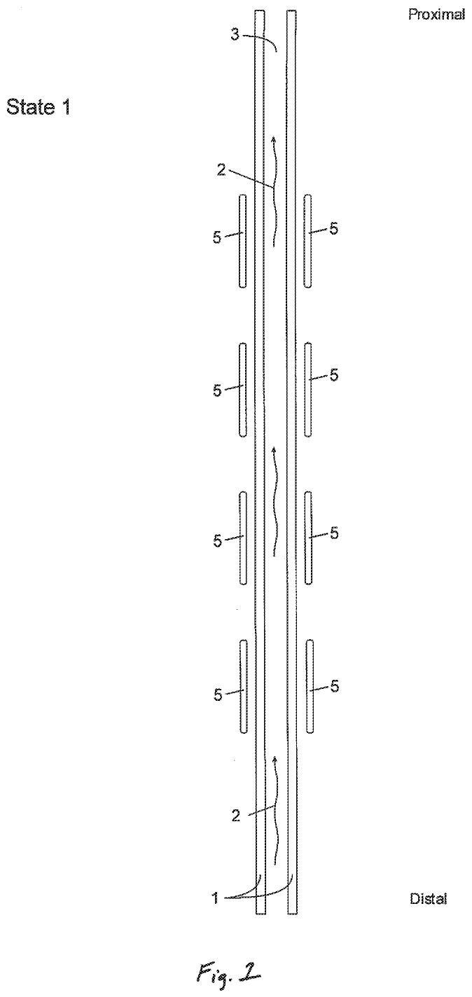

As a description of both exemplary methodology and systems according to the present disclosure, reference is made to now FIG. 1, which is a stylized/schematic rendering of a blood vessel 4 with treatment pads 5 in proximity thereto. For purposes of illustration the blood vessel 4 is shown in the absence of muscle, bone, skin, and the like, though anatomically, skeletal muscle is employed via electrostimulation to, in turn, stimulate or pressurize blood vessels, particularly veins. The blood vessel 4 may be any blood vessel in the body but with respect to particular embodiments of the present disclosure, the blood vessel 4 is a large vein in the foot, leg, hand, or arm, such as a tibial or saphenous vein. It is known that certain large veins within the human body have one-way valves as a part of the anatomy. In some respects, in a healthy individual, such veins can serve to eliminate or reduce “retrograde” flow of blood through the veins which would be in a distal direction, as blood normally travels in an artery. It should be appreciated that methods of the present disclosure provide therapeutic effect with respect to action upon particular veins whether such veins are those having internal valves or not. Hence, such valves are not illustrated in FIGS. 1-7.

Anatomically, the interior layer of the vein 4 is the endothelial layer 1. This is the innermost layer of a vein that is in actual contact with blood flow 2 and defines the inner flexible lumen 3 of the vein 4. The influence of the endothelium is far reaching and is more than simply a conduit for blood. It is the largest organ in the body and would be equivalent in size to approximately six tennis courts if spread out. It exerts control over an array of mechanisms which serve to maintain vascular tone and blood fluidity by maintaining vascular smooth muscle tone, regulating angiogenesis and cell proliferation, mediating inflammatory and immune responses, regulating vascular permeability, regulating thrombolysis, regulating leukocyte adhesion, regulating platelet adhesion and aggregation, and regulating lipid oxidation, among other actions and effects.

The endothelium exerts such control through endocrine, paracrine and autocrine processes wherein the endothelial cells secrete vasoactive substances such as hormones, genes, proteins, transcription factors and others, resulting in the regulatory actions listed above. This group of events is generally known as, “endothelial mechanotransduction.” Mechanotransduction refers to the processes through which cells sense and respond to mechanical stimuli by converting them to biochemical signals that elicit specific cellular responses.

Endothelial mechanotransduction happens in response to blood flow and laminar shear stress, induced from the mechanical forces caused by the rubbing of blood cells on the endothelium (the lining of blood vessels). When people are young, the normal physiologic levels of blood flow and shear stress keep blood vessels (and the whole cardiovascular system) healthy. Later in life, people make diet and lifestyle choices that can lower blood flow, clog the blood vessels with fatty deposits and impair the regulatory processes necessary for vascular health. The endothelium can then become dysfunctional contributing to atherosclerosis (hardening of the arteries), diabetes, hypertension (high blood pressure), delayed wound healing, vasculitis, congestive heart failure, critical limb ischemia, neuropathy and more.

Methods of the present disclosure positively affect the endothelium as well as treating sepsis by improving vascular return of blood from the extremities of a patient. However, other benefits of aiding return of blood flow not directly related to the endothelium per se may also be observed. Thus, the present disclosure and the effects of the methods herein are not strictly limited to those that rely upon endothelial effects. Further, there may exist in the prior art certain devices and methods that can be observed to improve return blood flow and possibly endothelial function. However, in various embodiments, the present disclosure presents an improved “wave form” that can be applied to a plurality of treatment pads placed on one or more extremities that stimulate blood vessels and the endothelial layer 1 by utilizing the patient's own skeletal muscle as a “pump”. It has been known that such a pumping action is affected by normal movement of a person, particularly in walking, but one who is immobile or otherwise unable to tolerate walking, for example, does not derive the full benefit of this anatomical pump. This pumping action is employed as a treatment for, and preventative measure against, sepsis.

In accordance with embodiments of the present disclosure, electrical stimulation pads 5 may be applied in pairs on opposite sides of a patient's limb. Electrical stimulation applied to the skin can result in contraction of muscle tissue surrounding the vein and provide a pumping action according to the waveforms and methods herein. In reality, many blood vessels may run within any limb or extremity such that one or more veins receive the benefit of the stimulation described herein.

Distal and proximal ends are labelled in FIG. 1. In the case of application of the pads 5 to a patient's leg, the distal end represents the feet and the proximal end represents the upper thigh, for example. As shown in FIG. 1, the endothelial layer 1 and surrounding muscle are relaxed, blood flow 2 is weak through the lumen 3. Four pairs of treatment pads 5 are distributed along the limb from distal to proximal. Treatment pads 5 (also known as electrodes) may be applied to the patient either by self-adhesive means or straps or in a garment. In some embodiments, the pads 5 are applied in pairs, opposed 180° on the feet, calves, lower thighs, and upper thighs. The hands, forearms, biceps, and shoulders, provide other placement locations that may be acceptable and may achieve the desired results. In some embodiments, in order to achieve the desired therapeutic threshold of blood movement, a minimum of 4 pairs of pads on each extremity must be used.

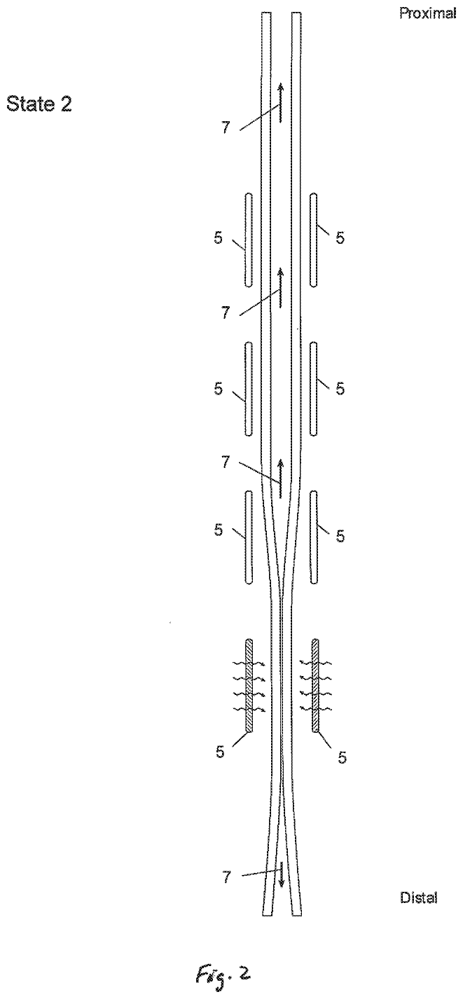

Referring now to FIG. 2, a schematic drawing showing the blood vessel 4 and pads 5 of FIG. 1 at initiation of a treatment sequence. Here, the most distal pair of pads 5 has been activated by application of current resulting in squeezing or closing of a portion of the lumen 3 by surrounding skeletal muscle. For purposes of the present disclosure, it is understood that voltage is also applied, and the particular relationship between applied voltage and applied current may rest upon a number of factors including the impedance of the pads 5 and the patient's body. In some embodiments, voltage may be applied to one pad out of a pair while the opposite pad acts as ground, or is supplied with a negative voltage thereby increasing current flow or voltage differential even further (within safe limits) while limiting the amount of voltage (positive or negative) applied to any single pad. In any event, blood flow 7 may be (or occur, or move) both proximal and distal at this stage, particularly if the vein 4 is a vein without anatomical valves or if the valves are weak or otherwise ineffective.

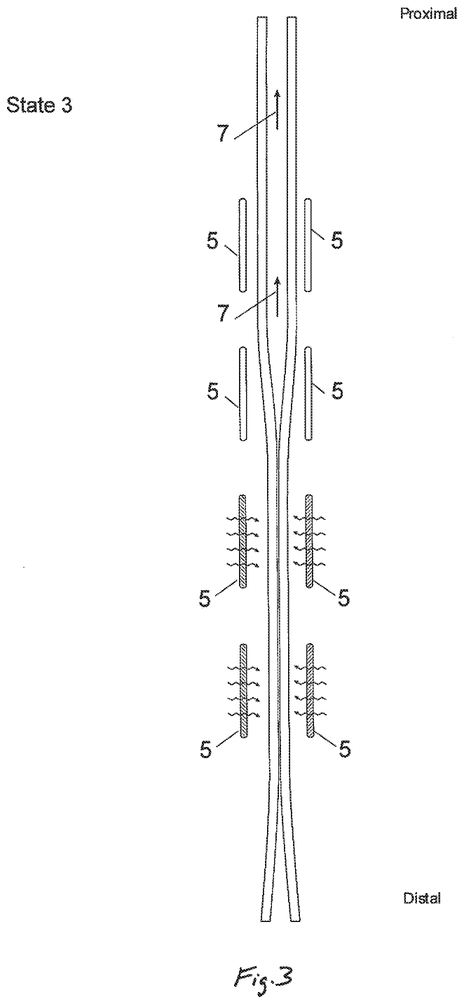

Referring now to FIG. 3 a schematic drawing showing the blood vessel 4 and pads 5 of FIG. 1 as treatment continues from FIG. 2 is shown. Here an overlapping, sequential protocol wave-form, state 3, of the present disclosure can being to be seen. The second most distal pair of pads 5 receive current causing muscle contractions which squeeze the blood vessel 4, closing the lumen 3 and forcing blood flow 7 from the area. None of the blood flow 7 is forced distally since the first pair of pads is still receiving current. Additionally, the blood flow 7 may be more forceful that that experienced at rest. Particularly if the patient is in ill health or non-ambulatory. The blood flow 7 is substantial enough to induce shear stress and activation of the endothelial layer 1 as discussed herein.

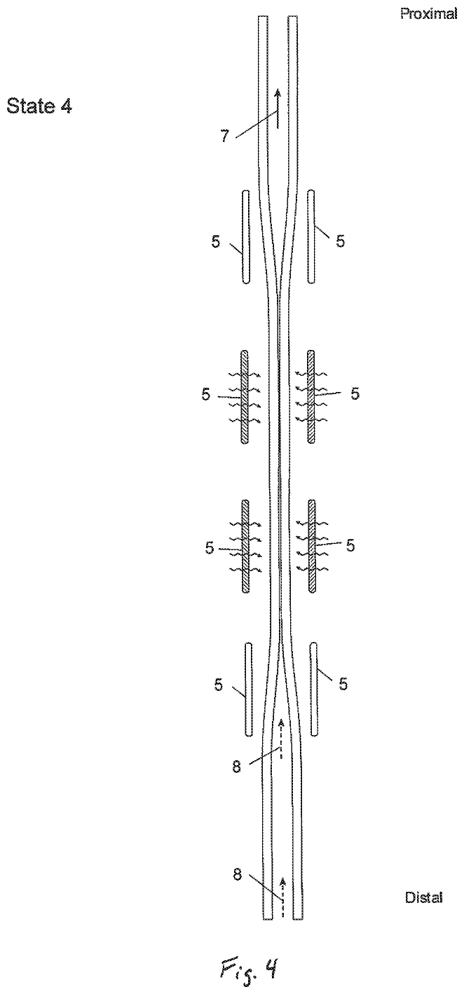

Referring now to FIG. 4, a schematic drawing showing the blood vessel and pads of FIG. 1 as treatment continues from FIG. 3 is shown. FIG. 4 shows the showing of the overlapping, sequential protocol, state 4. Here, the third most distal pair of pads 5 receives current causing muscle contractions which squeeze the blood vessel 4, closing the lumen 3 and forcing blood 7 further from the area (in the proximal direction). None, or at least very little, of the blood is forced distally since the second most distal pair of pads 5 (adjacent in the distal direction) is still receiving current. Current to the first pair of pads activated is terminated causing the blood vessel 4 to be allowed to expand and begin drawing refill blood 8 into the lumen 3.

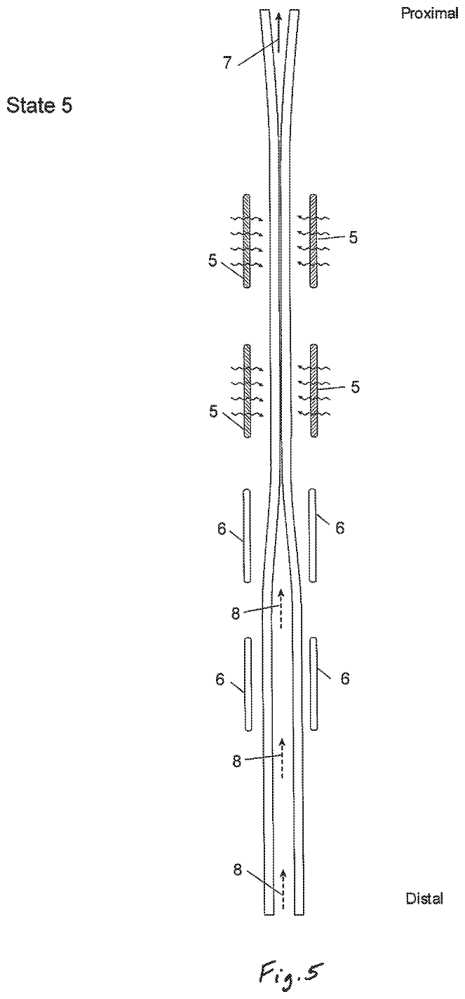

Referring now to FIG. 5, a schematic drawing showing the blood vessel 4 and pads 5 of FIG. 1 as treatment continues from FIG. 4 is shown. FIG. 5 shows the overlapping, sequential protocol, state 5. The fourth most distal pair of pads 5 receive current causing muscle contractions which squeeze the blood vessel 4, closing the lumen 3 and forcing blood 7 from the area further proximally. Again, little or none of the blood 7 is forced distally since the third most distal pair of pads is still receiving current. Current to the second pair of pads (second most distal and also second activated) is terminated after activation of most proximal pair of pads the allowing the blood vessel 4 to expand even further toward the proximal direction and continue to draw refill blood 8 deeper into the lumen 3.

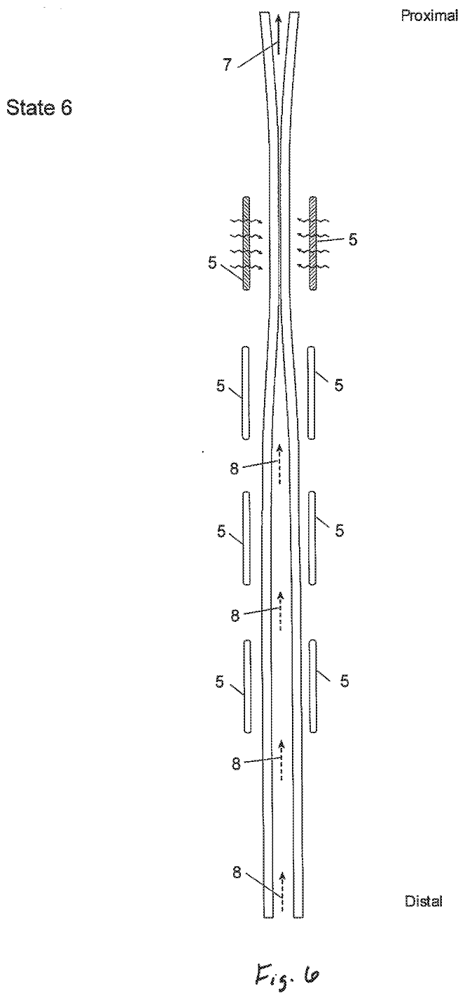

Referring now to FIG. 6, a schematic drawing showing the blood vessel 4 and pads 5 of FIG. 1 as treatment continues from FIG. 5 is shown. FIG. 6 shows the overlapping, sequential protocol, state 6. Current to the third pair of pads 5 (third distally and also third activated) is terminated allowing the blood vessel 4 to expand and continue drawing refill blood 8 deeper into the lumen 3.

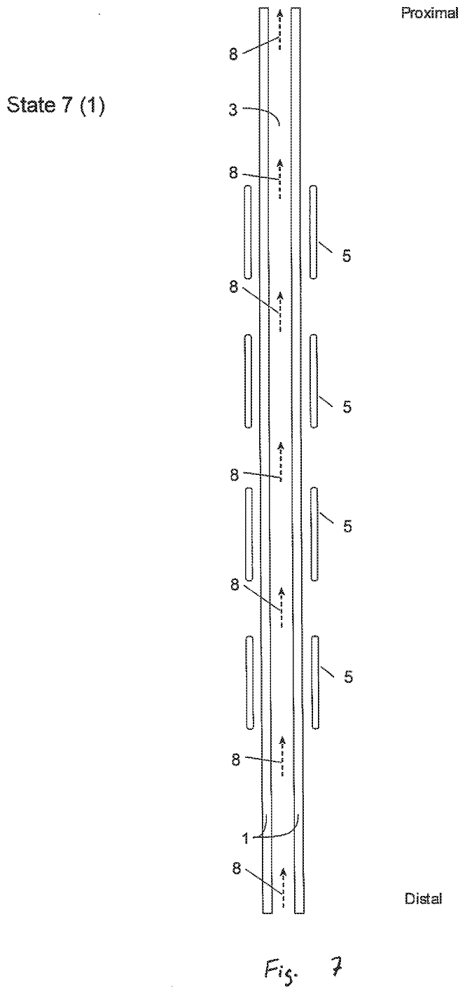

Referring now to FIG. 7, is a schematic drawing showing the blood vessel 4 and pads 5 of FIG. 1 as treatment continues from FIG. 6. Current to the fourth pair of pads 5 (most proximal) is terminated allowing the blood vessel 4 to re-expand along the entire length of the treatment draw refill blood 8 deeper into the lumen 3. This illustrate state, following application of a full wave form through the full set of pads 5 is substantially similar to state 1, FIG. 1. However, blood flow 8 is moving with more force than before (e.g., more forcefully than blood flow 2, FIG. 1). This is the major result of overlapping, sequential timing and the plurality of treatment pads according to embodiments of the present disclosure.

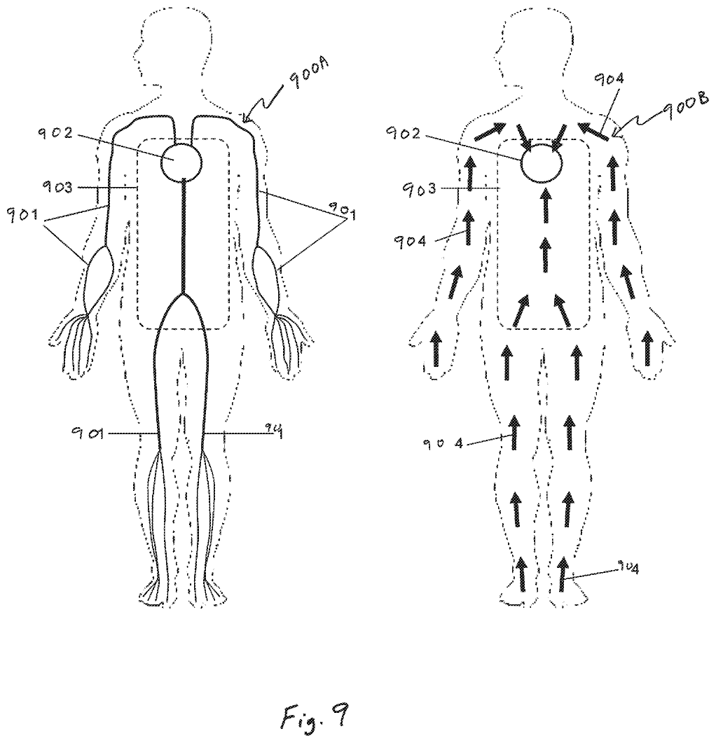

Although the sequence of FIGS. 1-7 illustrates a treatment mode employing four pairs of pads 5, it should be understood that more or fewer pairs of pads 5 may be employed. However, the overlapping aspects of the treatment wave form method would require at least two pairs of pads. Additionally, four pairs as shown provide sufficient stimulation of muscles along a limb so as to enhance proximal blood flow from an extremity to the patient's heart. This is called venous return, and results, according to the Frank-Starling law in higher preload and stroke volume. The wave-form method thus raises cardiac output which in many disease states (such as sepsis) is highly desirable (see, e.g., FIG. 9). If, for some reason, further stimulation points are desired, more than four pairs of pads 5 may be provided and it may be possible to activate a second wave-form before the first has completed (if sufficient distance has been provided between them that there is sufficient return blood flow 8 to be “pushed” by a second wave-form).

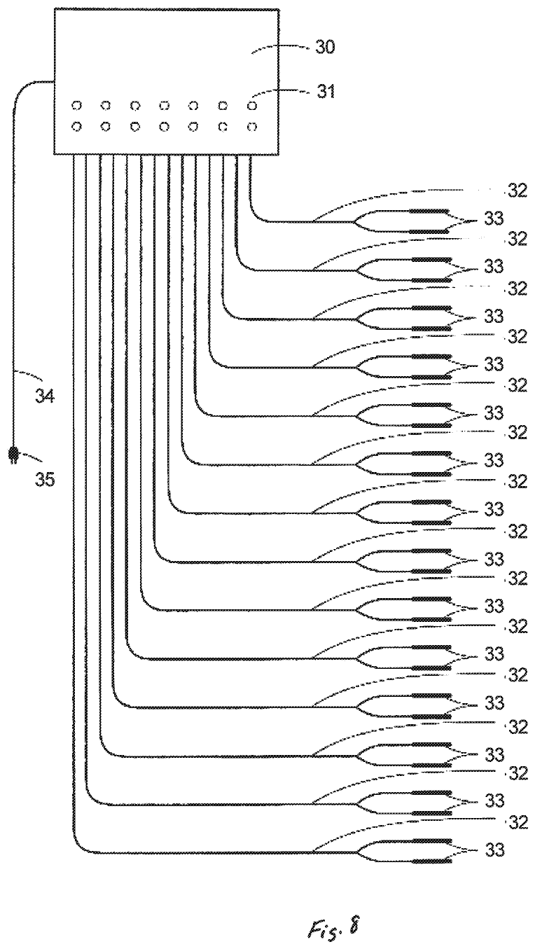

Referring now to FIG. 8, a drawing of a wave-form stimulation device 30 for providing treatments according to aspects of the present disclosure is shown. The wave-form stimulation device may be, in effect, a signal generator. Thus, it may include all necessary hardware and controls as are known in the art to safely apply various electrical signals, currents and voltages that are therapeutic yet safe for the human body. The wave-form stimulation device 30 comprises a plurality of leads 32. Each lead 32 attaches to a pair of treatment pads 33 (corresponding to the treatment pads 5 of FIGS. 1-7). An electrical cord 34 and plug 35 for alternating current (AC) input from a wall socket is provided. The treatment device 30 contains the necessarily internal hardware to convert the AC power to direct current (DC) for safe application to the patient via the leads 32 and pads 33. A number of controls 31 including necessary knobs, dials, levers, switches, and the like are provided to enable the operating therapist to control current/voltage applied within safe but therapeutically effective parameters.

In some embodiments, the leads 32 may be divided into groups of four, such that four pairs of pads may be applied to an extremity or limb of a patient. The number of leads 32 may vary. In some embodiments, at least 8 pairs of leads are provided such that both arms or both legs of a patient may have at least four pairs of pads applied in sequence. In another embodiment, 16 pairs of leads are provided such that both arms and both legs may be provided with four pairs of leads and all extremities be subject to the therapeutic application of the electrical wave-forms discussed herein.

Referring now to FIG. 9, a stylized representation of the human vascular system 900A is shown (left) along with a stylized representation of blood flow 900B that may result from applications of systems and methods of the present disclosure. Blood vessels, more particularly veins 901, carry blood back to the heart 902. Blood flow leaves the heart 902 and flows into the body core 903 then into the limbs. The arrows 904 represent blood flow which is increased by neuromuscular stimulation according to systems and methods of the present disclosure.

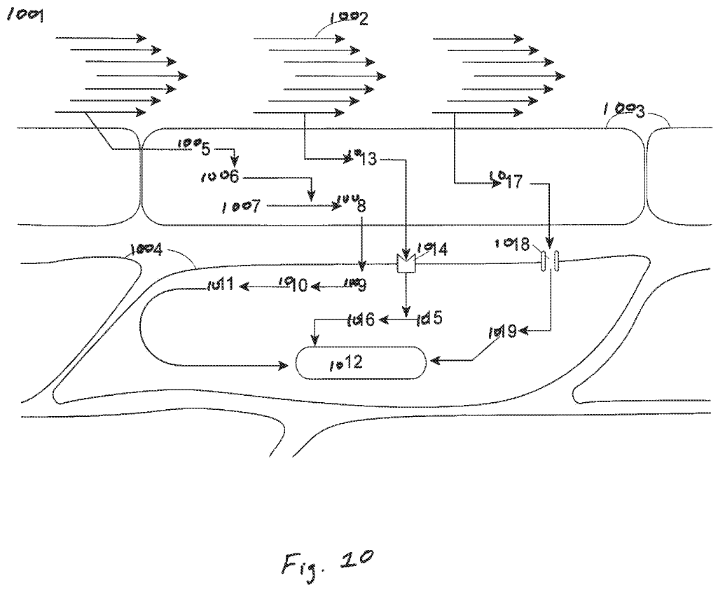

Referring now to FIG. 10, three exemplary endothelial mechanotransduction-generated changes in body chemistry via autocrine and paracrine processes based on systems and methods of the present disclosure are shown. Blood 1001 flows, aided by muscular contraction according to the present disclosure as shown by flow arrows 1002 through the vascular lumen 1004 creating shear stress with the frictional drag on the surface of endothelial cells 1003. A signal elicited by the friction travels down a calcium pathway 1005 to some endothelial nitric oxide synthase (eNOS) 1006. The eNOS synthesizes nitric oxide (NO) 1008 from L-arginine 1007. The NO immediately moves into the adjacent smooth muscle cell 1004 where it causes soluble guanylate cyclase 1009 to reduce to guanylate triphosphate 1010, then guanylate monophosphate 1011 which causes relaxation (noted at 1012) of the smooth muscle cells 1004 resulting in vasodilation.

Another signal elicited by the friction from blood flow 1002 travels down another pathway where it elevates prostacyclin 1013. The prostacyclin (PGI2) travels to a receptor 1014 on the surface of a smooth muscle cell 1004. Once it enters the smooth muscle cell, the PGI2 reduces adenosine triphosphate (ATP) to cyclic adenosine monophosphate (cAMP) 1016, which causes relaxation (noted at 1012) of the smooth muscle cells 1004 resulting in vasodilation.

Shear stress also sends a signal down a pathway into an endothelial cell 1003 where it upregulates endothelium derived hyperpolarizing factor 1017, which sends a signal through open potassium channels 1018, into the smooth muscle cell 1004 causing hyperpolarization 1019, and relaxation 1012 of the smooth muscle cells 1004 resulting in vasodilation.

These are three examples of the processes which are replicated throughout the body resulting in significant changes to the body's chemistry such that sepsis or septic shock can be avoided or reversed. Generally speaking, these processes move the vascular system toward homeostasis while low flow/shear stress exacerbates many, if not most, of the problems of sepsis/septic shock.

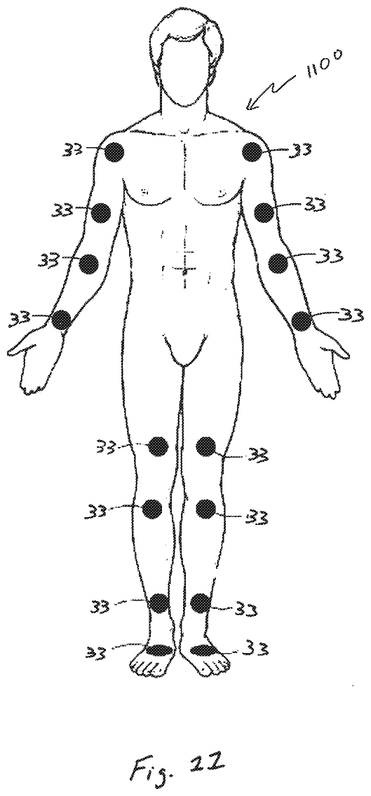

Referring now to FIG. 11, a stylized representation of a human body 1100 shows approximate placement of treatment pads 33 (electrodes) for the stimulation to be applied according to aspects of the present disclosure. Treatment pads 33 may be applied in pairs on opposite sides of the limbs, thus the body 1100 may have a complementary pad 33 on the posterior side of the patient apart from every pad 33 illustrated. In other embodiments, the pads 33 may be applied on complementary left and ride sides of the limbs though application nearer the more developed skeletal muscle planes may result in increased effect. As shown and described with respect to FIG. 8, pads 33 connect to leads 32 and then to the waveform generation device 30.

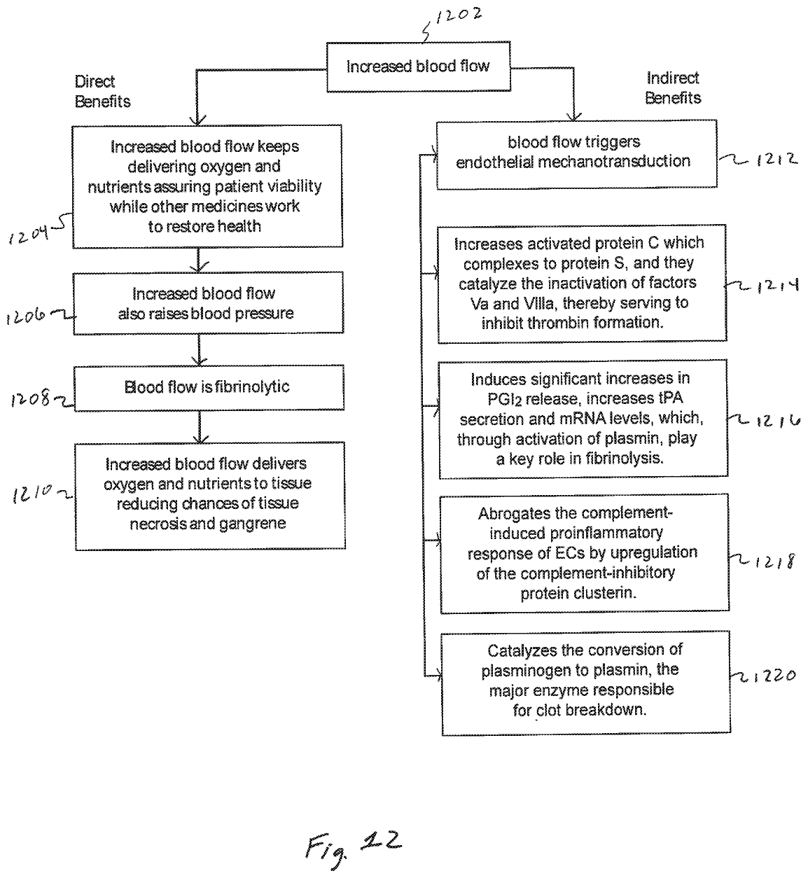

Referring now to FIG. 12 a flow chart 1200 showing various aspects of the results of using systems and methods of the present disclosure to increase blood flow (in addition to treatment/avoidance of sepsis/septic shock). Direct and indirect benefits of the enhanced blood flow are shown. Increased blood flow according to systems and methods of the present disclosure 1202 provide direct benefits including delivery of oxygen, nutrients, medicines etc. within the body as shown at 1204. Blood pressure may also be raised 1206 as well as effecting fibrinolytic blood flow at 1208. Chance of gangrene and necrosis is also reduced as shown at 1210. It should be understood that these direct benefits are not an exhaustive list, nor do they necessarily occur in any particular order, or all at once.

To further elaborate on direct benefits, movement of blood is useful following surgery, either by electric stimulation or pneumatic compression, to block thrombus development, blood movement being thrombolytic in nature. Various embodiments of the present disclosure use a plurality of electric treatment pads 33 on all four extremities to maximize blood movement and help restore the body's natural thrombolytic processes. The increased muscle contractions and blood flow increase blood pressure which is crucial to recovery. The increased blood flow helps deliver more oxygen and other nutrients to not only keep the organs viable, but also to raise tissue oxygen levels throughout the body and thereby counteract the effects of DIC and prevent gangrene in the extremities. By artificially restoring blood flow according to the present disclosure, when needed, the current systems and methods enlarge the window of opportunity to let the antibacterial and anti-inflammatory medications do their jobs. This could extend possible treatment duration by hours or days depending upon the patient's condition and the efficacy of treatment.

Indirect benefits include endothelial mechanotransduction at 1212. Activated protein C may be increased as shown at 1214 with its resultant benefits including catalysis of factors Va and VIIIa and thrombin inhibition. PGI₂ release at 1216 increases t-PA secretion and mRNA levels, which may play a key role in fibrinolysis by plasmin activation. Complement-induced proinflammatory response of ECs may be abrogated by upregulation of the complement-inhibitory protein clusterin as shown at 1218. Conversion of plasminogen to plasmin may be catalyzed as shown at 2020. Here again, It should be understood that these indirect benefits are not an exhaustive list, nor do they necessarily occur in any particular order, or all at once.

To further elaborate on the indirect benefits, increased blood flow increases endothelial shear stress which, in turn, triggers a series of physiologic events collectively known as endothelial mechanotransduction, bringing about a cascade of changes to the patient's blood chemistry. The physiologically most important activator of intravascular fibrinolysis may be tissue-type plasminogen activator (t-PA). The endothelium synthesizes and stores t-PA and the shear stress dependent release of the enzyme is an important protective response to prevent thrombus formation. Shear stress abrogates the complement-induced proinflammatory response of endothelial cells by upregulation of the complement-inhibitory protein clusterin; decreases endothelial cell tissue factor activity by raising the secretion of tissue factor pathway inhibitor; and catalyzes the conversion of plasminogen to plasmin, the major enzyme responsible for clot breakdown; Increases activated protein C which complexes to protein S, and they catalyze the inactivation of factors Va and VIIa, thereby serving to inhibit thrombin formation; and helps restore the body's natural shear-dependent anticoagulant cascades that can reduce endothelial cell dysfunction by rendering the cells less responsive to inflammatory mediators, facilitate the neutralization of inflammatory mediators and decrease loss of endothelial barrier function.

The equation for blood pressure is CO×SVR=BP, or, Cardiac Output times Systemic Vascular Resistance equals Blood Pressure. Another equation is VR=CO, Venous Return equals Cardiac Output. Therefore, as shear stress therapy as described herein elevates Venous Retur, the blood pressure is similarly elevated. In simpler terms, the muscle contractions created by stimulation are a form of exercise, and it is a well-known fact that exercise elevates blood pressure. This may eliminate the need for vasopressors and help salvage limbs, stop or reverse sepsis and septic shock, and a host of other positive events.

### * * * *

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

### * * * * *

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A method of treatment of sepsis in a patient in need of such treatment comprising:
applying a first plurality of pairs of electric treatment pads to a first limb of the patient from a distal to a proximal location on the first limb; and
providing an electrical neuromuscular stimulation to the first plurality of pairs of treatment pads according to a waveform;
wherein:
the waveform is applied in a sequential and overlapping manner to the first plurality of pairs of treatment pads such that the electrical neuromuscular stimulation progresses from the distal to the proximal location on the first limb;
the waveform activates a first most distal pair of pads of the first plurality of treatment pads and thereafter activates a second most distal pair of pads of the first plurality of treatment pads while keeping the first most distal pair of pads of the first plurality of treatment pads activated; and
the waveform deactivates the first most distal pair of pads of the first plurality of treatment pads when a third most distal pair of pads of the first plurality of treatment pads is activated.

2. The method of claim 1, further comprising:
applying a second plurality of pairs of electric treatment pads to a second limb of the patient from a distal to a proximal location on the second limb; and
providing the electrical neuromuscular stimulation to the second plurality of pairs of treatment pads according to the predetermined waveform;
wherein:
the waveform is applied in a sequential and overlapping manner to the second plurality of pairs of treatment pads such that the electrical neuromuscular stimulation progresses from the distal to the proximal location on the second limb;
the waveform activates a first most distal pair of pads of the second plurality of treatment pads and thereafter activates a second most distal pair of pads of the second plurality of treatment pads while keeping the first most distal pair of pads of the second plurality of treatment pads activated; and
the waveform deactivates the first most distal pair of pads of the second plurality of treatment pads when a third most distal pair of pads of the second plurality of treatment pads is activated.

3. The method of claim 2, further comprising:
applying a third plurality of pairs of electric treatment pads to a third limb of the patient from a distal to a proximal location on the third limb; and
providing the electrical neuromuscular stimulation to the third plurality of pairs of treatment pads according to the predetermined waveform;
wherein:
the waveform is applied in a sequential and overlapping manner to the third plurality of pairs of treatment pads such that the electrical neuromuscular stimulation progresses from the distal to the proximal location on the third limb;
the waveform activates a first, most distal pair of pads of the third plurality of treatment pads and thereafter activates a second most distal pair of pads of the third plurality of treatment pads while keeping the first most distal pair of pads of the third plurality of treatment pads activated; and
the waveform deactivates the first most distal pair of pads of the third plurality of treatment pads when a third most distal pair of pads of the third plurality of treatment pads is activated.

4. The method of claim 3, further comprising:
applying a fourth plurality of pairs of electric treatment pads to a fourth limb of the patient from a distal to a proximal location on the fourth limb; and
providing the electrical neuromuscular stimulation to the fourth plurality of pairs of treatment pads according to the predetermined waveform;
wherein:
the waveform is applied in a sequential and overlapping manner to the fourth plurality of pairs of treatment pads such that the electrical neuromuscular stimulation progresses from the distal to the proximal location on the fourth limb;
the waveform activates a first most distal pair of pads of the fourth plurality of treatment pads and thereafter activates a second most distal pair of pads of the fourth plurality of treatment pads while keeping the first most distal pair of pads of the fourth plurality of treatment pads activated; and
the waveform deactivates the first most distal pair of pads of the fourth plurality of treatment pads when a third most distal pair of pads of the fourth plurality of treatment pads is activated.

5. The method of claim 1, wherein the first limb is an arm.

6. The method of claim 1, wherein the first limb is a leg.

7. The method of claim 2, wherein the first limb and second limb are arms.

8. The method of claim 2, wherein the first limb and second limb are legs.

9. The method of claim 2, wherein the first limb is a leg and the second limb is an arm.

10. The method of claim 4, wherein the first and second limbs are left and right arms, respectively, and the third and fourth limbs are left and right legs, respectively.

11. The method of claim 10, wherein the waveform is applied to the first and third plurality of treatment pads simultaneously, followed by application of the waveform to the second and fourth treatment pads simultaneously.

12. The method of claim 11, wherein application of the waveform to the first and third plurality of treatment pads does not overlap with application of the waveform to the second and fourth plurality of treatment pads.

13. The method of claim 1, wherein the electrical neuromuscular stimulation at each pair of the first plurality of pairs of treatment pads is about 500 ms in duration.

14. A method of treatment of a patient in need of a treatment for sepsis comprising:
applying a first plurality of electrically conductive treatment pads along plurality of locations along a first limb of the patient in need of treatment, the locations along the first limb being from distal to proximal; and
providing an electrical waveform as an application of current to the first plurality of electrically conductive treatment pads;
wherein the electrical waveform is applied in a sequential and overlapping manner to the first plurality of treatment pads such that an electrical neuromuscular stimulation progresses from the distal to proximal locations on the first limb;
wherein the electrical waveform applies the electrical neuromuscular stimulation such that stimulation occurs at two adjacent locations on the first limb, except for a beginning of the waveform when only a most distal location of the first limb receives stimulation and an end of the waveform when only a most proximal location on the first limb receives stimulation; and
wherein the electrical waveform maintains stimulation at no more than two adjacent locations on the first limb at any time.

15. The method of claim 14 comprising:
applying a second plurality of electrically conductive treatment pads along plurality of locations along a second limb of the patient in need of treatment, the locations along the first limb being from distal to proximal; and
providing the electrical waveform as an application of current to the second plurality of electrically conductive treatment pads;
wherein the electrical waveform is applied in the sequential and overlapping manner to the second plurality of treatment pads such that an electrical neuromuscular stimulation progresses from the distal to proximal locations on the second limb;
wherein the electrical waveform applies the electrical neuromuscular stimulation such that stimulation occurs at two adjacent locations on the second limb, except for a beginning of the waveform when only a most distal location of the second limb receives stimulation and an end of the waveform when only a most proximal location on the second limb receives stimulation; and
wherein the electrical waveform maintains stimulation at no more than two adjacent locations on the second limb at any time.

16. The method of claim 15, wherein the first limb is an arm of the patient and the second limb is a leg of the patient.

17. The method of claim 16, wherein the electrical waveform is applied to the plurality of electrically conductive treatment pads on the first limb and to the plurality of electrically conductive treatment pads on the second limb simultaneously.

18. A device for treatment of a patient in need of a treatment for sepsis comprising:
an electrical waveform generator;
a first plurality of pairs of electric treatment pads adapted to attach to a first limb of the patient from a distal to a proximal location on the first limb, each of the pairs of electrical treatment pads of the first plurality of pairs of electrical treatment pads, when active, providing electrical neuromuscular stimulation at their respective location to the first limb of the patient; and
leads electrically connecting the first plurality of pairs of electric treatment pads to the waveform generator;
wherein the electrical waveform generator provides a stimulation waveform to the first plurality of pairs of electrical treatment pads;
wherein the stimulation waveform activates the first plurality of pairs of treatment pads in a sequential and overlapping manner such that the electrical neuromuscular stimulation progresses from the distal to the proximal location on the first limb;
wherein the stimulation waveform activates a first most distal pair of pads of the first plurality of treatment pads and thereafter activates a second most distal pair of pads of the first plurality of treatment pads while keeping the first most distal pair of pads of the first plurality of treatment pads activated; and
the stimulation waveform deactivates the first most distal pair of pads of the first plurality of treatment pads when a third most distal pair of pads of the first plurality of treatment pads is activated.

19. The device of claim 18 further comprising:
a second plurality of pairs of electric treatment pads adapted to attach to a second limb of the patient from a distal to a proximal location on the second limb, each of the pairs of electrical treatment pads of the second plurality of pairs of electrical treatment pads, when active, providing electrical neuromuscular stimulation at their respective locations to the second limb of the patient; and
leads electrically connecting the second plurality of pairs of electric treatment pads to the waveform generator;
wherein the electrical waveform generator provides the stimulation waveform to the second plurality of pairs of electrical treatment pads;
wherein the stimulation waveform activates the second plurality of pairs of treatment pads in a sequential and overlapping manner such that the electrical neuromuscular stimulation progresses from the distal to the proximal location on the second limb;
wherein the stimulation waveform activates a first most distal pair of pads of the second plurality of treatment pads and thereafter activates a second most distal pair of pads of the second plurality of treatment pads while keeping the first most distal pair of pads of the first plurality of treatment pads activated; and
the stimulation waveform deactivates the first most distal pair of pads of the second plurality of treatment pads when a third most distal pair of pads of the second plurality of treatment pads is activated.

20. The device of claim 19, wherein the waveform generator provides the stimulation waveform to the first and second pluralities of treatment pads simultaneously.
